(19) United States
(12) Patent Application Publication
(10) Pub. No.: US 2016/0022409 A1
(43) Pub. Date: Jan. 28, 2016

(54) ENDOVASCULAR PERFUSION STENT GRAFT

(71) Applicant: University Hospitals of Cleveland, Cleveland, OH (US)

(72) Inventors: Alon S. Aharon, Spokane, WA (US); Donald M. Voltz, Twinsburg, OH (US)

(21) Appl. No.: 14/876,334

(22) Filed: Oct. 6, 2015

Related U.S. Application Data
(63) Continuation-in-part of application No. 13/839,794, filed on Mar. 15, 2013.

(51) Int. Cl. A61F 2/06; A61F 2/24

(52) U.S. Cl. CPC A61F 2/064; A61F 2/06; A61F 2/24; A61F 2/2412; A61F 2002/068

(57) ABSTRACT

An endovascular perfusion stent graft is provided that can include a graft device, a balloon and a valve. The graft device can be inserted into a vessel. The balloon can be inserted into the graft device and can temporarily restrict passage of fluid or gas received via a perfusion port when the balloon is inflated via a bulb that provides air pressure to the balloon. The perfusion port can be operatively connected to another perfusion port configured to allow the fluid or the gas to pass through another balloon. The valve can be attached to the graft device.

### CROSS REFERENCE TO RELATED APPLICATIONS

This application is a continuation-in-part of, and claims priority to, U.S. patent application Ser. No. 13/839,794, filed on Mar. 15, 2013, and entitled “ENDOVASCULAR PERFUSION STENT GRAFT”. The entirety of the foregoing application is hereby incorporated by reference.

### TECHNICAL FIELD

The subject disclosure relates to a stent graft and, also generally, to an endovascular perfusion stent graft.

### BACKGROUND

Every day, medical teams perform hundreds of surgeries, including heart surgeries. Such procedures can be scheduled or, in some cases, the procedures can be performed during an emergency situation. As one example, heart surgery can involve the repair of the heart due to aortic dissection, which is a separation of the aorta walls. The separation (or tear) can start relatively small; however, the tear can become larger. Aortic dissection can cause bleeding into and along the wall of the aorta, which is the major artery that carries blood away from the heart.

Conventional heart surgery procedures to repair an aortic dissection include cardiopulmonary bypass due to the difficulty of operating on a beating heart. During the cardiopulmonary bypass, and while the patient is undergoing surgery, the patient can be cooled (e.g., the patient's body temperature can be brought down to, and maintained at, around 18 degrees Celsius). The patient's blood is drained to a heart lung machine reservoir in order for the surgery to be performed in a relatively bloodless manner. Consequently, the patient's brain (as well as the entire body) is not receiving blood flow, at least for some period of time. Therefore, the surgeon must operate quickly in order to insert a graft and sew the graft in place. After the graft is installed, blood can be transferred from the heart lung machine and back into the patient.

Surgery to repair an aortic dissection can be difficult to perform since the surgeon is operating deep within the patient's chest. Further, the aorta is friable (e.g., similar to tissue paper) and may be dissected when the inside of the aorta is completely split from the muscular aortic wall (creating two channels of blood flow) and/or a total aortic arch repair might be needed. Such repairs can include surgery to perform a distal aortic anastomosis. The friable nature of the aorta can, therefore, compound surgery complexity, as well as increase the length of surgery.

### SUMMARY

A simplified summary is provided herein to help enable a basic or general understanding of various aspects of example, non-limiting embodiments that follow in the more detailed description and the accompanying drawings. This summary is not intended, however, as an extensive or exhaustive overview. Instead, the sole purpose of this summary is to present some concepts related to some example, non-limiting embodiments in a simplified form as a prelude to the more detailed description of the various embodiments that follow. It is also noted that the detailed description may include additional or alternative embodiments beyond those described in this summary.

An aspect provided herein relates to a system that can include a graft device configured to be inserted into a vessel. The system can also include a balloon configured to temporarily restrict passage of fluid or gas through the vessel when the balloon is temporary inserted into the graft device. Further, the system can include a perfusion port configured to selectively allow the passage of the fluid or the gas through the balloon and to the vessel.

In an example, the graft device can include an inner surface and an outer surface. Further to this example, the balloon can be further configured to expand within the graft device. The balloon can contact the inner surface of the graft device and the outer surface of the graft device can contact an inner wall of the vessel.

In another example, the graft device can include at least one expandable portion configured to extend outwardly for contact with an inner wall of the vessel. Further, to this example, the at least one expandable portion can extend a length of the graft device.

According to another example, at least a portion of an outer surface of the graft device can include a connection mechanism. Further to this example, the connection mechanism can include one or more of a plurality of hook structures, a sealant material strip at a distal end of the graft, and an adhesive strip at a distal end of the graft. Additionally or alternatively, the connection mechanism can extend at least partially around a periphery of the graft device at one or more locations.

In another example, the system can include a bulb configured to provide air pressure to the balloon through a balloon infusion port.

According to another example, the system can include another graft device configured to be inserted into another vessel. Also included in the system can be another balloon configured to temporarily restrict the passage of the fluid or the gas through the another vessel when the another balloon is temporary inserted into the another graft device. Further, the system can include another perfusion port operatively connected to the perfusion port and configured to allow the passage of the gas or the fluid through the another balloon. Further to this example, the another vessel can be at least a portion of the vessel.

In an implementation, the vessel can be one of an ascending aorta, a descending aorta, or an arch of an aorta.

Another aspect provided herein relates to a method that can include inserting a graft device into a vessel and deploying an expandable device into the graft device. The method can also include causing the expandable device to push the graft device outward and pushing the graft device into contact with an inner wall of the vessel.

In an example, the method can include inserting a perfusion port through the expandable device. Further to this example, the method can include causing fluid to flow through the perfusion port and the expandable device.

Further to the above example, the perfusion port can include a first end located on a first side of the expandable device and a second end located on a second side of the expandable device. The method can include attaching another vessel to a fluid collection device and attaching the fluid collection device to the first end of the perfusion port. The method can also include allowing the fluid to flow from the fluid collection device to the first end, through the expandable device, and out the second end to the another vessel.

According to another example, the method can include inserting another graft device into another vessel and deploying another expandable device into the another graft device. Further to this example, the method can include causing the another expandable device to push the another graft device outward and pushing the another graft device into contact with the inner wall of the another vessel.

In accordance with another example, the method can include inserting a perfusion port through the expandable device and causing a fluid to flow through the perfusion port and through the expandable device. Further to this example, the method can include inserting another perfusion port through the another expandable device and attaching a first end of the perfusion port to another first end of the another perfusion port. Further, the method can include causing the fluid to flow from the first end of the perfusion port to the another first end of the another perfusion port.

In another example, the method can include unrolling a first end of the graft device. Further to this example, the method can include contacting the first end of the graft device with at least a portion of the vessel.

In still another example, the graft device can include at least one connector. Further to this example, pushing the graft device into contact with the inner wall of the vessel can include attaching the at least one connector to the inner wall of the vessel.

Another aspect provided herein relates to a graft device that can include a graft configured to provide a conduit for blood flow and a stent configured to provide structure support to the graft and push the graft toward a vessel. The graft device can also include at least one fastener configured to securely anchor the graft to the vessel as the graft contacts the vessel.

In an example, the at least one fastener can include a plurality of hook structures located at one or more locations on an outer surface of the graft device. In a further example, the at least one fastener can include a sealant material strip or an adhesive strip at a distal end of the graft.

Another aspect provided herein relates to a system that can include a valve. The valve can be attached to the graft device.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

Various non-limiting embodiments are further described with reference to the accompanying drawings in which:

FIG. 1 illustrates a schematic representation of a normal, healthy aorta and its associated branching arteries;

FIG. 2 illustrates schematic representations of example aortic dissections in various locations of the aorta;

FIG. 3 illustrates a system, according to an aspect;

FIG. 4 illustrates another embodiment of the system of FIG. 3, according to an aspect;

FIG. 5 illustrates a further embodiment of the system of FIG. 3, according to an aspect;

FIG. 6 illustrates another embodiment of the system of FIG. 3, according to an aspect;

FIG. 7 illustrates an example, non-limiting embodiment of a graft device that can be utilized with the disclosed aspects;

FIG. 8 illustrates a device used to repair an aortic arch, according to an aspect;

FIG. 9 illustrates another example of repair of an aortic arch, wherein the three main vessels are dissected and reimplanted, according to an aspect;

FIG. 10 illustrates another embodiment of a graft device, according to an aspect;

FIG. 11 illustrates a non-limiting embodiment of a valve, according to an aspect;

FIG. 12 illustrates a method for aortic dissection repair, according to an aspect;

FIG. 13 illustrates another method for aortic dissection repair, according to an aspect; and

FIG. 14 illustrates yet another method for aortic dissection repair, according to an aspect.

### DETAILED DESCRIPTION

Aspects of the subject disclosure will now be described more fully hereinafter with reference to the accompanying drawings in which example embodiments are shown. In the following description, for purposes of explanation, numerous specific details are set forth in order to provide a thorough understanding of the various embodiments. However, the subject disclosure may be embodied in many different forms and should not be construed as limited to the example embodiments set forth herein.

The aspects disclosed herein allow for the construction of a distal aortic anastomosis under direct vision (e.g., by the surgeon) in a short amount of time using a distal anastomotic system. In an implementation, the device comprises fastening means, such as a sealant material strip(s) or an adhesive strip(s), that provide a secure anastomosis between the diseased and highly friable aorta and the graft. Further, with the disclosed aspects, the body can be continually perfused via a second arterial channel through the inflated balloon, which can mitigate systemic circulatory arrest. The disclosed aspects can be used in many types of aortic pathology including ascending aneurysm repair, and so forth.

Although the various aspects are described with reference to the treatment of aortic dissections, the disclosed aspects are not limited to aortic dissections. Instead, it is to be appreciated that the various aspects can be implemented for various diseases and causes and aortic dissection is merely one example.

Aortic dissection is a common catastrophic event involving the aorta, which is an artery carrying blood from the heart to the body. An aortic dissection represents a pathologic state in which a tear occurs in the intima of the aortic wall, diverting blood flow. Such a tear effectively splits the aorta in two, similar to peeling away layers of an onion. Dissections can proceed in the aorta for variable lengths, shearing off side branches along the way. Usually, the entry tear is relatively short (e.g., around 0.5-3.0 cm long) and located at the proximal extent of the dissection. The resulting false lumen (e.g., abnormal channel within the wall of the involved artery) can extend distally as a consequence of blood shearing off the inner layers in an antegrade manner. Retograde dissections can also occur, but are less common. Over time, continuous blood flow beyond the arterial wall can cause the aorta to rupture. Patients with acute dissection might be close to death (e.g., within an hour or two) and, therefore, timely intervention is necessary in order to achieve the best results for both short-term and long-term recovery.

The main classification system for aortic dissection is the Stanford system. A Stanford type “A” dissection occurs proximal to the left subclavian artery. A Stanford type “B” dissection occurs distal to the left subclavian artery. Type A dissections tend to occur more frequently in younger patients while type B dissections tend to occur more frequently in older patients. The type A dissection is a surgical emergency. Surgical mortality for acute type A aortic dissection is around 7 to 30%. Mortality can be more than 45% in octogenarians and can be more than 60% in unstable patients. Predictors of operative mortality and morbidity include age, cardiac tamponade, hypotension, acidosis, renal failure, neurovascular embarrassment, myocardial ischemia, and congestive heart failure (CHF).

Some intraoperative predictors of poor outcome include transverse arch replacement and progression of aortic dissection into the descending aorta. Another predictor of poor outcome is a prolonged circulatory arrest time (e.g., cerebral, systemic). Other predictors of poor outcome include a prolong operative time, as well as massive transfusion requirements.

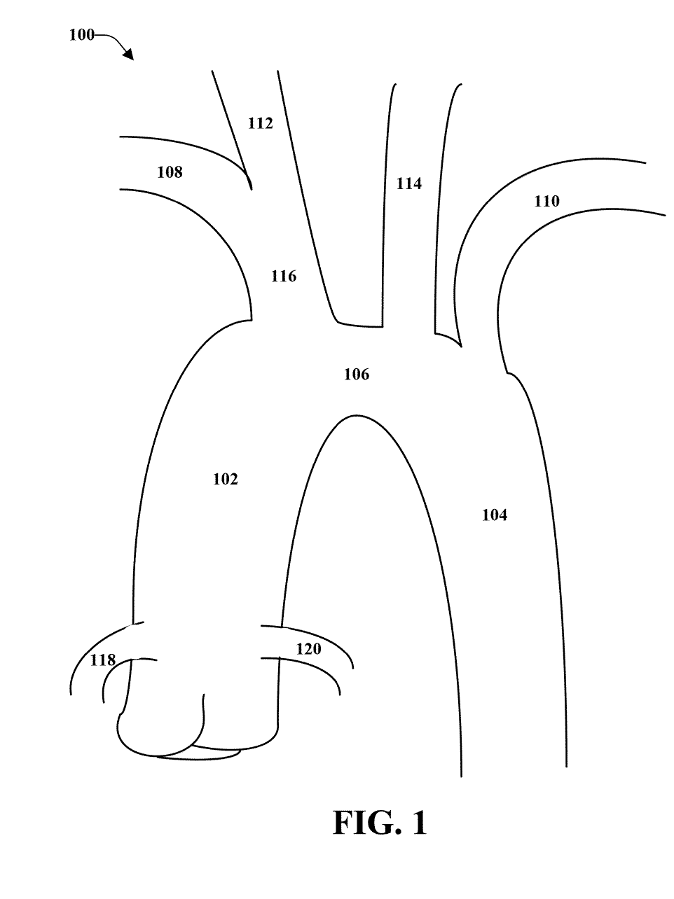

FIG. 1 illustrates a schematic representation of a normal, healthy aorta 100 and its associated branching arteries. It is noted that the figures provided herein are not drawn to scale and are simplified for purposes of explaining the various aspects.

The aorta 100 comprises an ascending aorta 102 and a descending aorta 104. An arch 106 of the aorta 100 connects the ascending aorta 102 and the descending aorta 104. A right subclavian artery 108 and a left subclavian artery 110 receive blood from the top (e.g., arch 106) of the aorta 100. The right subclavian artery 108 supplies blood to the right arm and the left subclavian artery 110 supplies blood to the left arm.

A right common carotid artery 112 and a left common carotid artery 114 receive blood from the arch 106 and supply blood to the head and neck. An innominate artery 116 arises from the arch 106 and divides into the right subclavian artery 108 and the right common carotid artery 112.

Further, a right coronary artery 118 and a left coronary artery 120 branch from the aorta 100, close to its point of departure from the heart and carry oxygen-rich blood to the heart. The right coronary artery 118 supplies blood to the right upper chamber (atrium) of the heart and the left coronary artery 120 supplies blood to the left atrium of the heart.

When aortic dissection occurs, the inner lining of the aortic can tear (e.g., in the ascending aorta 102, in the descending aorta 104). Tearing of the inner lining can result in a false lumen, diverting the blood flow. The false lumen can extend distally due to the blood shearing off the inner layers in an antegrade fashion.

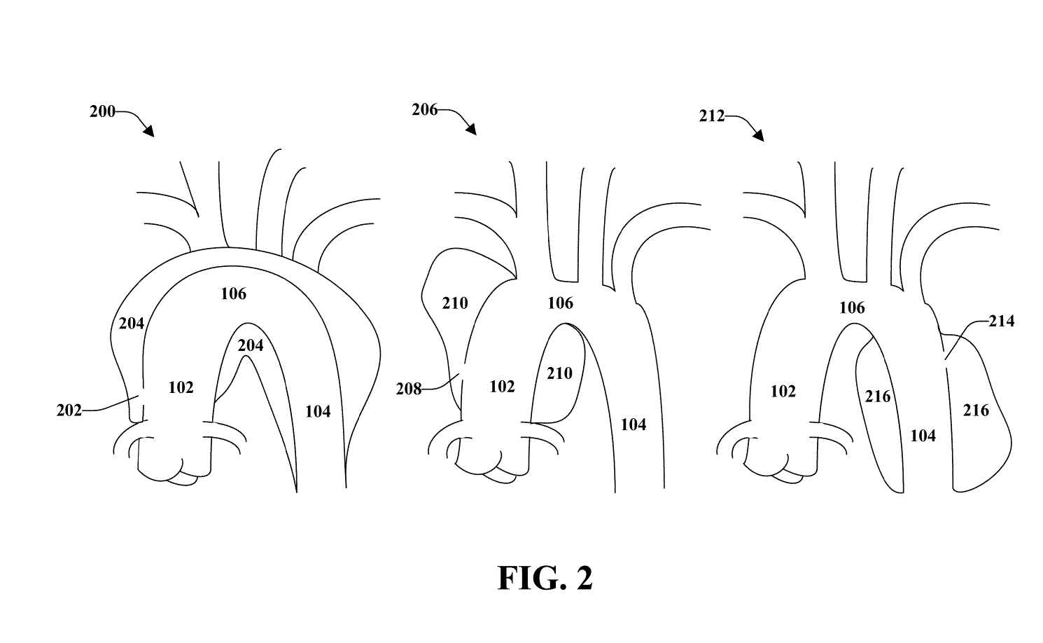

FIG. 2 illustrates schematic representations of example aortic dissections in various locations of the aorta. Aortic dissections can be ascending aortic dissections or descending aortic dissections. Aortic dissections can cause false lumens, which can fill with blood, diverting blood flow. Further, the false lumens can rupture, causing internal blood loss.

The first schematic illustration 200 depicts an ascending aortic dissection, shown as a tear 202 in the ascending aorta 102. In this situation, a false lumen 204 can encase the ascending aorta 102, the arch 106, and the descending aorta 104. The second schematic illustration 206 of FIG. 2 depicts another example ascending aortic dissection. In this case, there is a tear 208 in the ascending aorta 102 however, a false lumen 210, forms only around the ascending aorta 102. The third schematic illustration 212 of FIG. 2 depicts a descending aortic dissection. Here, there is a tear 214 in the descending aorta 104. In this situation, a false lumen 216 can surround the descending aorta 104.

As a brief example, a conventional surgical technique to repair an aortic dissection will now be described. It is noted that other variations for repairing aortic dissections could be performed and the following description is simply an example of a simple procedure.

At the beginning of surgery, institution of cardiopulmonary bypass can be established through a right axillary artery cannulation and a sternotomy incision. For axillary artery cannulation, the incision can be just under the right clavicle. The sternotomy incision can be through the midline of the sternum.

The patient is cooled (e.g., the temperature of the patient is reduced) and, using various techniques, exsanguinated into a heart lung machine so that the distal aortic anastomosis can be performed in a relatively bloodless field. During the exsanguination, all the patient's blood is drained into the heart lung machine reservoir while the patient is maintained at about 18 degrees Celsius. Consequently, the brain (as well as other parts of the body) is not receiving any blood flow until the distal (e.g., the graft to distal aortic anastomosis-side) is completed.

The distal anastomosis may entail extensive dissection and complete reimplantation of the great vessels into the graft as an island of tissue or separately. The three great vessels referred to here are the three vessels that come off of the aortic (e.g., the innominate artery 116, the left common carotid artery 114, the left subclavian artery 110, of FIG. 1)

The graft is then implanted into the distal aorta. The graft is sewn proximally to the aortic, just above the coronary arteries (e.g., right coronary artery 118 and left coronary artery 120 of FIG. 1). If necessary, the entire aortic root may need to be replaced and the coronary arteries re-implanted into the graft. For example, this might be necessary if the dissection involves the aortic root (e.g., the aortic valve and the coronary arteries).

The distal anostomis is hand sewn and, therefore, can be very difficult to perform since the surgeon is operating deep in the left chest from the midline sternotomy incision. In addition, the aorta is very friable (e.g., similar to tissue paper) and may be completely dissected when the inside of the aorta (the intima) is completely split from the muscular aortic wall. This creates two channels of blood flow, which needs to be put back together during construction of the distal anastomosis.

To overcome various problems associated with conventional techniques, the disclosed aspects provide a stent graft to construct the anastomosis. Further, the stent graph disclosed herein can simplify operations, including operations to repair the aortic dissections.

As discussed above, when conventional techniques are used, the body and brain receive no blood flow during the distal anatomosis between the graft and thoracic aorta (e.g., aorta in the left chest). However, with the disclosed aspects, a distal anastomatic device can be deployed using a fairly rigid balloon. The distal anastomosis is thus completed quickly, under direct vision, by inflating the balloon and deploying a distal anastomotic system (e.g., a connection mechanism).

The balloon is kept inflated during the surgery and a perfusion catheter can be used. The perfusion catheter can be passed through the center of the balloon and can be connected to the arterial inflow line from the heart lung machine (or from an outlet port (e.g., vessel) of the body that supplies blood to the heart lung machine). Thus, the body receives systemic perfusion of cold blood (distal to the inflated balloon) and is not exposed to a period of circulatory arrest. Circulatory arrest can result in significant end-organ damage (e.g., renal failure, liver insufficiency, and so forth) and is a reason for poor outcome in high risk cases.

Additionally, since the axillary artery is cannulated, according to an aspect, the brain can also be perfused during the entire surgery with continuous cold blood. Specifically, blood flow through the arterial inflow line passes into the axillary artery and then into the inominate artery and onward via the right carotid artery to the brain. The base of the inominate artery can be clamped and blood flow goes toward the head. In an implementation, two arterial inflow lines can be used, one for the head and one for the body.

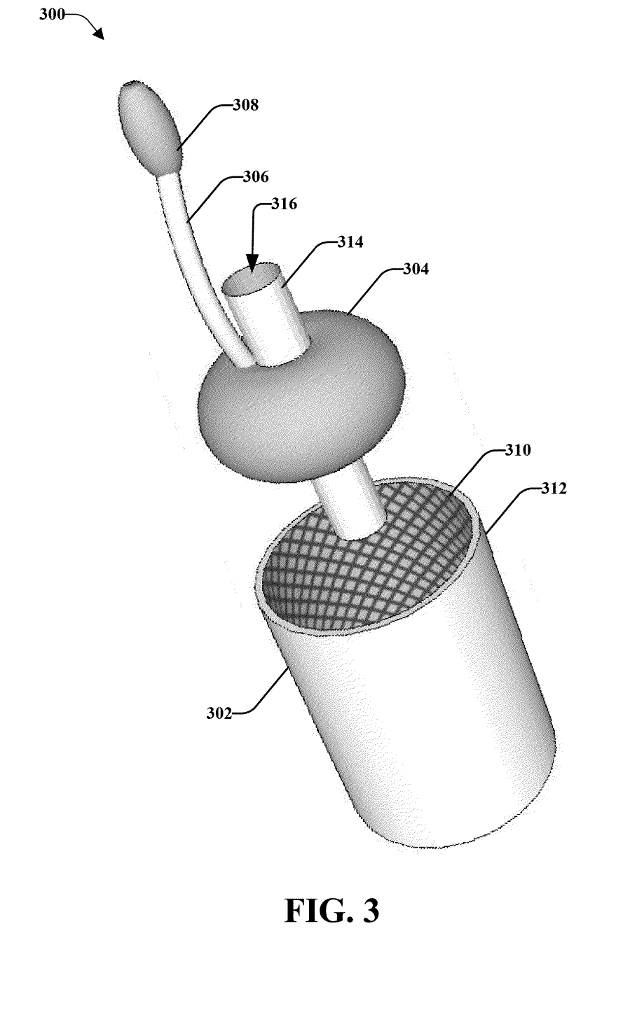

FIG. 3 illustrates a system 300, according to an aspect. System 300 can include a graft device 302 configured to be inserted into a vessel for deployment therein. For example, the graft device 302 can be inserted into an artery to repair an aortic dissection, according to an aspect. However, the disclosed aspects are not limited to this implementation and the system 300 can be utilized for other procedures, such as, for example, thoracic and abdominal aortic aneurysms, and so forth. For example, in operation, the graft device 302 can be placed in an artery, a vein, or other channel (e.g., intestines, windpipes, and so forth) within a human or animal body. In an example, a medical professional can place the graft device 302 in a desired location.

The graft device 302 can be constructed of fabric supported by a rigid structure. The fabric, for example, can be mesh material or another material that provides a conduit for blood flow. In an implementation, the graft device 302 can be a graft stent. For example, the graft stent can comprise a graft that provides the conduit for blood flow and the stent can anchor the graft to the aorta. Further, the stent can provide structure support for the graft material. In an example, the graft can be constructed of woven polyester or expanded polytetrafluoroethylene (ePTFE), or another material known in the art. The stent can be constructed of nitinol, stainless steel, or another material known in the art.

The apparatus also includes a balloon 304 that can be configured to be temporary inserted into the graft device 302. For example, an inflation/deflation line or a balloon infusion line 306 can be attached to the balloon 304 and can be configured to fill the balloon 304 with a gas, such as air, or a liquid, for example. The balloon 304 can be expanded (and/or deflated) by a bulb 308 operatively attached to the balloon infusion line 306. The bulb 308 can be squeezed by a single hand, wherein squeezing of the bulb 308 causes the balloon 304 to expand within the graft device 302.

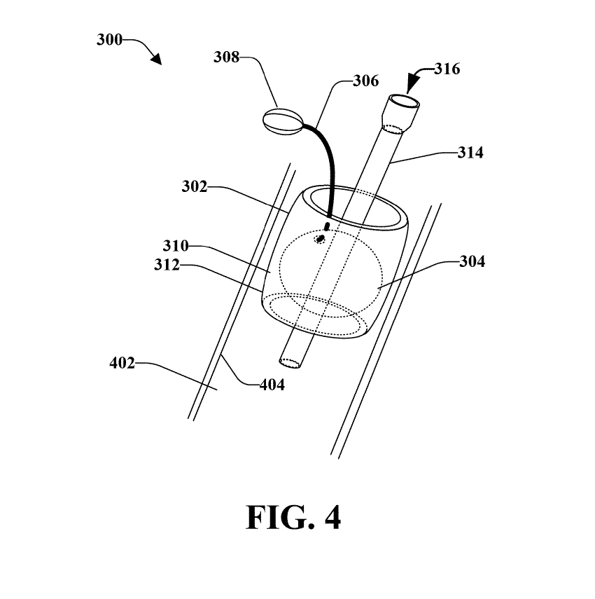

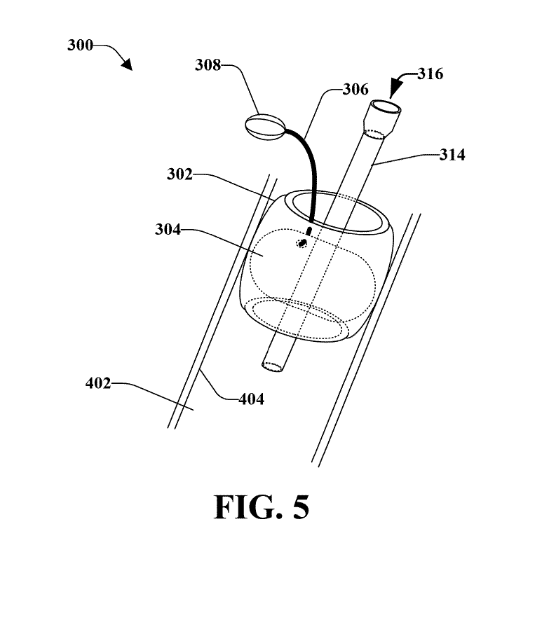

With reference now also to FIG. 4, the balloon 304 can be in inserted into the graft device 302 when the tubular element is located within a vessel 402 (e.g., a vein, an artery, and so on). Squeezing of the bulb 308 can cause air, for example, to traverse the balloon infusion line 306 and fill the balloon 304 with air (in this example). As the balloon is expanded within the graft device 302, the balloon can come into contact with an inner surface 310 of the graft device 302. This contact places pressure on the graft device 302, which causes expansion of the graft device 302 toward the inner wall 404 of the vessel 402, as illustrated in FIG. 5.

Thus, graft device 302 can be expandable and can be in a compressed state when inserted into vessel 402. Graft device 302 can then be expanded outwardly and distally, as illustrated in FIG. 5. The balloon 304 can be pressed against the graft device 302, which presses against an inner wall 404 of the vessel 402 such that fluid (e.g., blood) or gas (e.g., air from breathing) cannot pass through the area where the graft device 302 and the inner wall 404 contact.

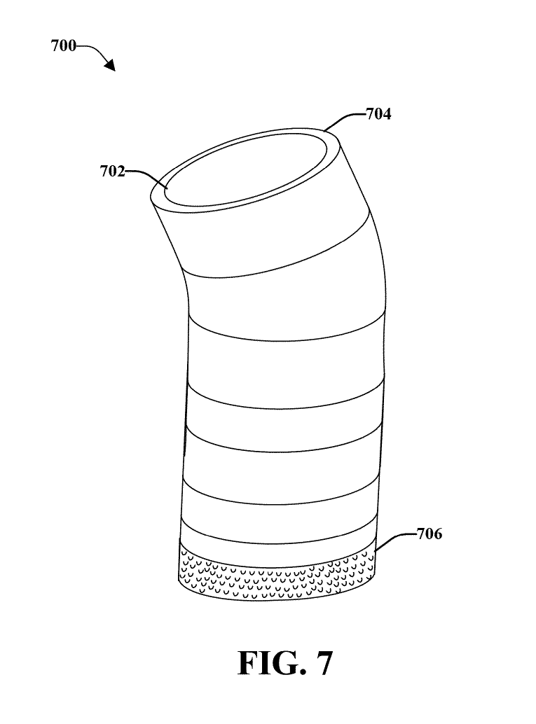

As the graft device 302 is expanded, an outer surface 312 comes into contact with an inner wall 404 of the vessel 402. As the outer surface 312 contacts the inner wall 404, a connection mechanism can be deployed to engage and secure the graft device 302 to the vessel 402. Further details related to the connection mechanism will be described in connection with FIG. 7 below.

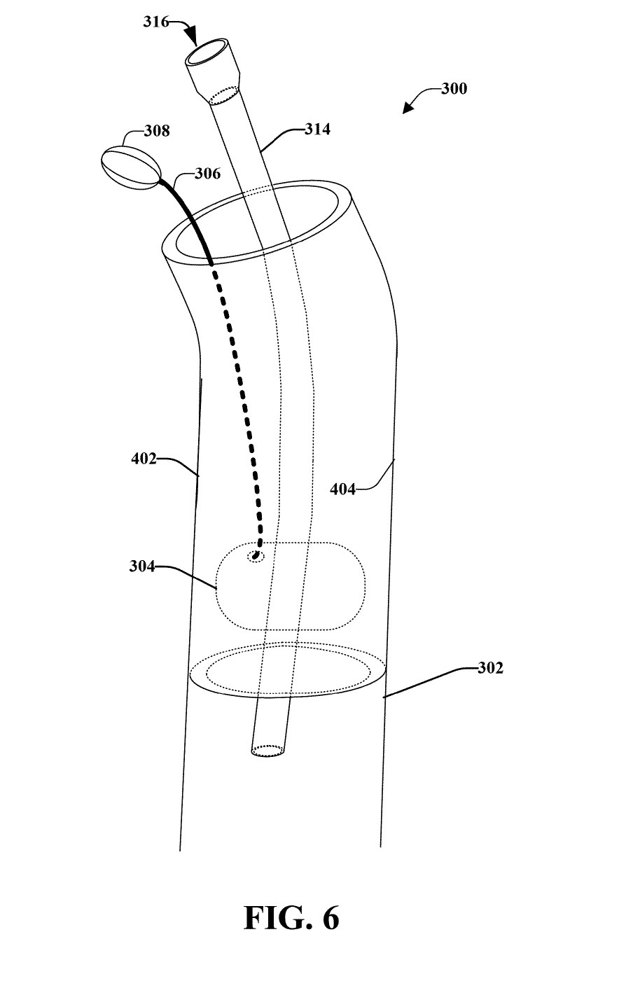

At about the same time as the graft device 302 is secured to the vessel 402, the balloon can be slightly deflated, and the balloon 304 removed from the graft device 302, as illustrated in FIG. 6. The balloon 304 can also be removed completely from the vessel 402. It is noted that although the balloon 304 is removed, the graft device 302 remains in secure engagement with the vessel 402. In an example, the balloon 304 can be deflated by squeezing the bulb 308, however, other mechanisms of releasing pressure can be utilized with the pump system (e.g., balloon 304, balloon infusion line 30, and bulb 308 combination).

With continuing reference to FIGS. 3-6, the system 300 can also include a perfusion catheter or perfusion port 314. Perfusion port 314 can be connectable to a perfusion device (e.g., a heart and lung machine). Perfusion port 314 can be connected before or after placement of the balloon 304 into the graft device 302. In an implementation, the perfusion port 314 can be inserted at substantially the same time as the balloon 304 is inserted into the vessel 402. According to an implementation, perfusion port 314 can be integrated with balloon 304 (e.g., one piece). However, according to other implementations, perfusion port 314 and balloon 304 are two pieces and the balloon 304 operates as a gasket to prevent leakage of fluids or gases between the perfusion port 314 and the balloon 304.

The perfusion port 314 can be in fluid communication with a perfusion device and a blood source. For example, an opening 316 in (e.g., through the middle of) the perfusion port 314 can allow blood to flow, perfusing the head and/or the body. The balloon 304 can operate as a clamp to stop the blood from flowing outward (e.g., on the side of the balloon 304 that is connected to the balloon infusion line 306). At about the same time as the balloon 304 is to be deflated, fluid entering the perfusion port 314 can be temporarily stopped until the surgery is completed. The perfusion port 314 can be removed from the vessel 402 at about the same time as the balloon 304 is removed from the vessel.

FIG. 7 illustrates an example, non-limiting embodiment of a graft device 700 that can be utilized with the disclosed aspects. Graft device 700 can be, for example, graft device 302 of FIGS. 3-6. As illustrated graft device 700 comprises an inner surface 702 (e.g., inner surface 310 of FIG. 3) and an outer surface 704 (e.g. outer surface 312 of FIG. 3).

At least a portion of the outer surface 704 of the graft device 700 can comprise a connection mechanism 706. The connection mechanism 706 is configured to quickly engage the vessel in a secure manner. Thus, the connection mechanism 706 can eliminate, or significantly reduce, the need to sew the graft device 700 into place, which reduces the length of the surgery. For example, anastomotic device (e.g., graft device 700) can comprise a series of very small hooks that can be deployed when the balloon is inflated. The hooks secure the stent graft to the native aorta, according to an aspect. The hooks can be formed of the same material as the graft device 700 or can be formed with different material that is compatible with and operatively connected to the graft device.

In another implementation, the connection mechanism 706 can be an adhesive strap and/or a sealant material strip. For example, the adhesive strip or sealant material strip can be located at a distal end of the graft device 700. However, according to other implementations, the adhesive strip or sealant material strip (or other type of connection mechanism) can be located at other positions on the outer surface 704.

As illustrated the connection mechanism 706 can be formed on a bottom section (e.g., section of the graft device 700 located farthest away from the pump assembly) of the graft device 700. However, the disclosed aspects are not limited to this implementation. Instead, the connection mechanism can cover all or substantially all of the outer surface 704 of the graft device 700. In another example, the connection mechanism 706 can be dispersed periodically or randomly over the outer surface 704 of the graft device 700. For example, a connection mechanism can be placed at one location around the perimeter of the graft device. In another example, two connection mechanisms can be placed at two different locations around the perimeter of the graft device. In a further example, three or more connection mechanisms can be placed at three or more locations around the perimeter of the graft device. Multiple connection mechanisms or fastening means can provide more opportunities for the graft device to securely engage with the vessel. However, one connection mechanism can be all that is necessary to securely engage the graft device with the vessel, according to an implementation.

Further, although the connection mechanism 706 is illustrated as encircling the graft device, the disclosed aspects are not so limited. Instead, a first connection mechanism can extend at least partially around a first half of the graft device and second connection mechanism can extend at least partially around a second half of the graft device. Further to this example, the first connection mechanism can be located at a bottom of the graft device (as illustrated) and the second connection mechanism can be located at a top of the graph device. In another example, multiple connection mechanisms can be located at various locations around the graphic device.

The connection mechanism 706 can comprise hook structures that are configured to “grab” or easily engage a member (e.g., a vessel). The hook structures can be strong enough to penetrate the vessel, at least slightly, without breakage of the hook structures. Further, the hook structures can pierce the vessel at about the same time as the graft device is brought into contact with an inner wall of the vessel and/or when the graft device is moved slightly within the vessel.

In another example, the connection mechanism 706 can comprise one or more adhesive strips and/or one or more sealant material strips. The one or more strips can be configured to security engage with the vessel at about the same time as the graph device is brought into contact with the inner wall of the vessel. In a further implementation, more than one type of connection mechanism 706 might be utilized with the graft device 706.

Further, graft device 700 (or graft device 302) can include at least one expandable portion configured to extend outwardly for contact with an inner wall of the vessel. The expandable portion can be located at the end (e.g., section of the graft device 700 located farthest away from the pump assembly) for extension thereof. The expandable portion can be configured to be rolled out, as necessary, to extend a length of the graft device. If a longer graft is not needed, the expandable portion can be retained in an unrolled state (or can be selectively removed from the graft device 700). According to an implementation, the expandable portion does not include connection mechanisms. However, according to another implementation, at least a portion of the expendable portion comprises one or more connection mechanisms.

As described above, the device (e.g., graft device 302, graft device 700) can be inserted into a vessel to allow a secure anastomosis between the diseased and highly friable aorta and graft. The disclosed aspects can also allow for construction of the distal anastomosis under direct vision (e.g., in a bloodless manner) and in a short amount of time (e.g., using the connection mechanism 706). Further, the patient can be continually perfused via a second arterial channel (e.g., perfusion port 314) through the inflated balloon, which can avoid, or significantly decrease, systemic circulatory arrest. Further, the disclosed aspects can be utilized in many types of aortic pathology including ascending aneurysm repair, and so forth.

As discussed, a Stanford type A dissection can occur just proximal to the left subclavian artery. The repair can require replacement of the proximal aorta, and possibly aortic root (aortic valve and coronary arteries) as well as aortic transverse arch in an open fashion (e.g., via a median sternotomy). At the end of an open repair, a dissection plane is present distal to the replaced aorta, which conventionally is left alone and not repaired. However, according to the disclosed aspects, the distal portion of the thoracic aorta (distal to the left subclavian artery) can be repaired by deploying a graft via the open aorta, down into the descending segment following open repair of the proximal segment.

The most difficult and highest risk type Stanford A repair is when the dissection includes the transverse aortic arch and descending aorta, such that the open repair requires replacing the descending aorta, transverse arch, and ascending aorta. When this occurs, the distal portion of the graft is sewn to the thoracic aorta in the left chest (the exposure is difficult as the approach is through a midline sternotomy). While the graft is being sewn to non-dissected aorta in the left chest, the body is cooled to about 18 degrees Celsius (in order to decrease metabolic needs). However, when this surgery is performed conventionally, the body does not receive any blood flow while the graft is being sewn to the descending aorta. This is one reason why many patients die during this procedure (e.g., kidney failure, liver failure, and so forth).

According to the disclosed aspects, the distal anastomosis can be performed using a graft device (e.g., graft device 302, graft device 700) that comprises one or more connection mechanisms (e.g., connection mechanism 706), which can allow for rapid completion of this stage of the operation. Further, continuous blood flow can be maintained through a perfusion catheter (e.g., perfusion port 314) connected to the arterial line of the bypass machine, thus maintaining blood flow to all organs.

Further, grafts, which are compressed or accordinated into the device can be deployed both down the thoracic aorta and proximally into the transverse arch and ascending aorta. Additional connections to the proximal aorta (aortic root and great vessels of the head) can be performed using the disclosed aspects (e.g., connection mechanism, hook structures, and so forth).

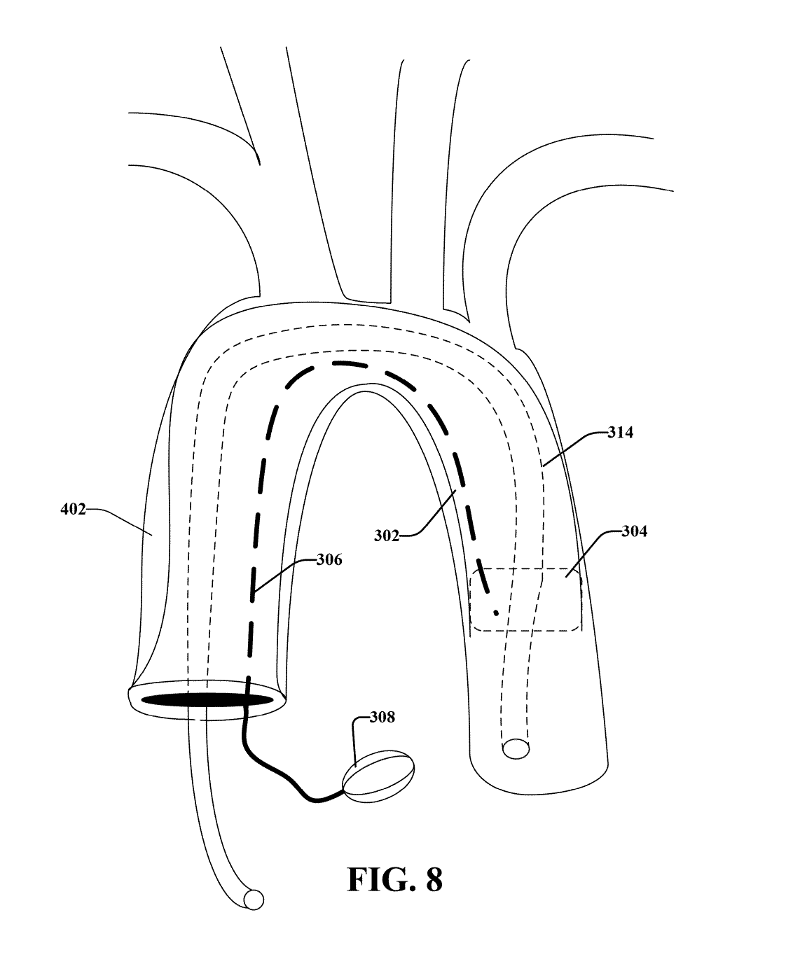

To further illustrate, FIG. 8 depicts the disclosed aspects used to repair an aortic arch, according to an aspect. It is noted that the same (or similar) element numbers as FIGS. 3-6 are used for this and the following figures for ease of understanding the disclosed aspects. In this case, the perfusion port 314 and balloon infusion port 306 are passed through an inner portion of the arch (e.g., conform to the configuration of the arch).

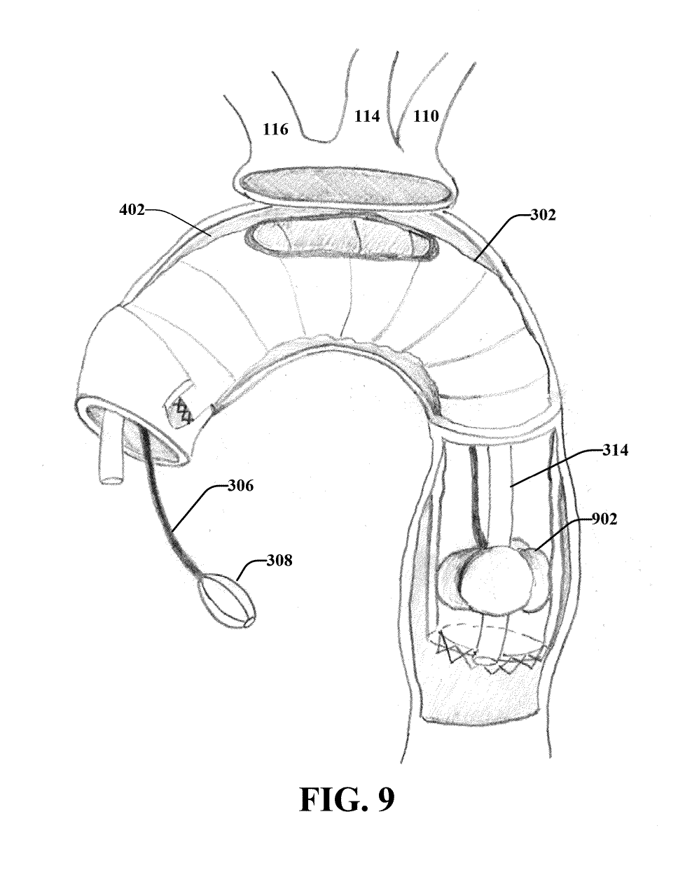

FIG. 9 illustrates another example of repair of an aortic arch, wherein the three main vessels are dissected and reimplanted, according to an aspect. As shown, the distal anastomosis may entail extensive dissection and complete reimplantation of the great vessels into the graft as an island of tissue or separately. The three great vessels referred to here are the three vessels that come off of the aortic (e.g., the innominate artery 116, the left common carotid artery 114, the left subclavian artery 110). As illustrated, according to some implementations, multiple balloons 902 can be utilized.

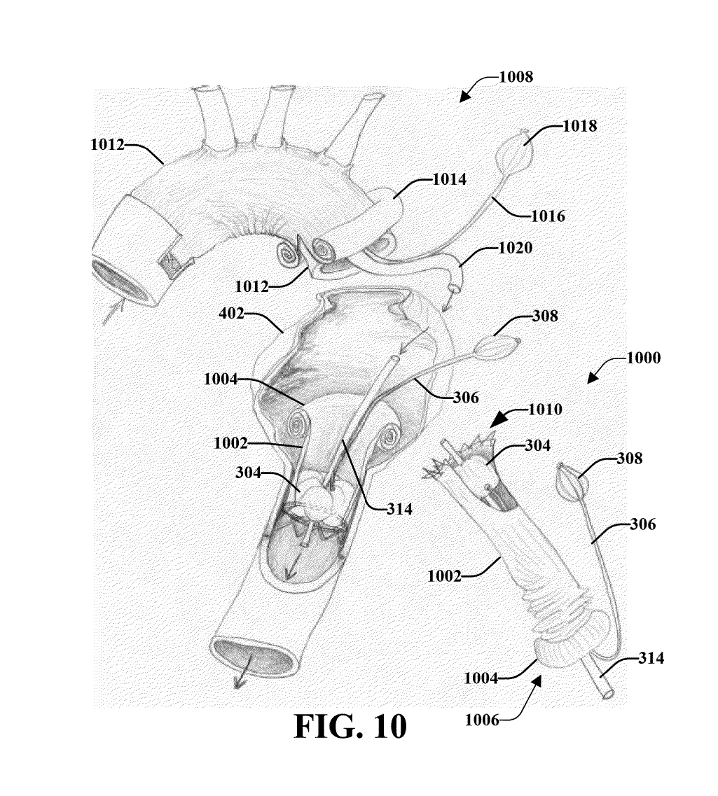

FIG. 10 illustrates another embodiment of a graft device 1000, according to an aspect. As illustrated, the graft device 1000 (depicted on the bottom right of the figure) comprises a graft 1002 that comprises excess material 1004 (e.g., an expandable portion) at a first end 1006 of the graft device. The graft device 1000 can be inserted into a vessel 402, as illustrated at 1008. A balloon 304 is configured to be inserted into the graft device 1000 and deployed at a second end 1010 of the graft device. The excess material 1004 can be unrolled, if necessary, to repair a larger area of the aorta. However, according to an implementation, one or more of the graft devices might not include the excess material, depending on the size and location of the repair.

Further, according to an implementation, a second graft device 1012 comprising excessive material 1014 can also be utilized during the surgery. To deploy the second graft device 1012, a second balloon (not shown), a second balloon infusion port 1016, and a second bulb 1018 can be utilized. The perfusion port 314 and a second perfusion port 1020 can be operatively connected to circulate blood through the body as the surgery is being performed (e.g., eliminate or reduce the use of a heart lung machine). For example, ends of the perfusion port 314 and the second perfusion port 1020 can be secured together with a clamp or another device that securely connects the ports. It is noted that the balloon 304 and a second balloon (disposed within the second graft device 1012) stop respective blood flow out of the vessels and into the operating area. The operation of the second balloon is substantially the same as the above figures and will not be repeated here.

Accordingly, one or more graft devices can be employed in a system comprising a plurality of perfusion ports (e.g., a plurality of perfusion catheters, a plurality of perfusion channels, etc.) and/or a plurality of balloons (e.g., a plurality of blocking balloons, a plurality of balloon blockers, sequential blocking balloons, etc.). The plurality of perfusion ports and/or the plurality of balloons can be inserted (e.g., deployed) into one or more vessels sequentially to allow distal perfusion while accomplishing vessel repair. It is to be appreciated that in certain implementations, a graft device can be associated with more than one balloon (e.g., more than one blocking balloon, more than one balloon blocker, etc.) and/or more than one perfusion port (e.g., perfusion catheter). In one example, a balloon (e.g., a blocking balloon, a balloon blocker, etc.) can be attached to another balloon (e.g., another blocking balloon, another balloon blocker, etc.) via a perfusion port. In another example, a first balloon can be attached to a first balloon infusion line and a second balloon can be attached to a second balloon infusion line. In yet another example, a first balloon and a second balloon can be attached to a corresponding (e.g., the same) balloon infusion line. It is also to be appreciated that a perfusion port (e.g., a perfusion catheter) can allow passage of another substance other than gas or fluid. For example, a perfusion port (e.g., a perfusion catheter) can be employed as a working port (e.g., a channel to allow further manipulation and/or passage of device(s) via the perfusion port). In certain implementations, a plurality of perfusion ports, a plurality of balloons, and/or graft device(s) as further described herein can be employed during a Bentall procedure (e.g., a Bentall cardiac surgery procedure).

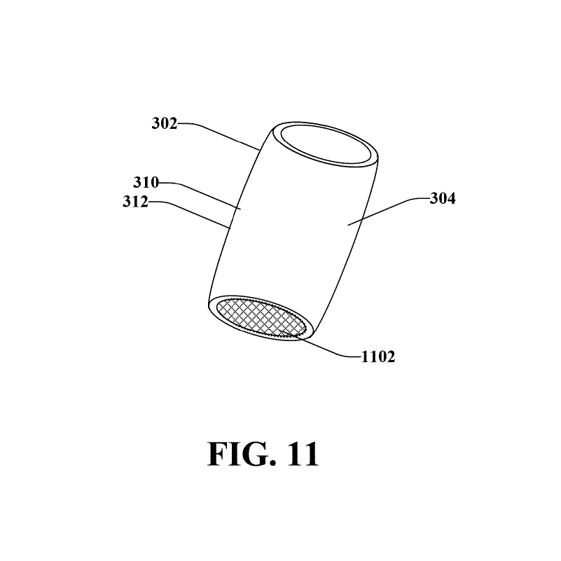

With reference now to FIG. 11, in an embodiment, the graft device 302 can additionally include a valve 1102. The valve 1102 can be attached to an end of the graft device 302 (e.g., the value 1102 can be attached to a distal end of the graft device 302). The valve 1102 can be configured to open and close to provide unidirectional flow of fluid or gas through the valve 1102. For example, the valve 1102 can be configured to open enough to allow a fluid or gas to flow through the valve 1102 in a particular direction (e.g., via the graft device 302 and/or a vessel) while also being configured to close to prevent fluid or gas from flowing through the valve 1102 in another direction (e.g., via the graft device 302 and/or a vessel). The valve 1102 can be configured to open and close based on pressure differences associated with the valve 1102. For example, the valve 1102 can be configured to open and close based on a fluid pressure difference associated with the valve 1102 or a gas pressure difference associated with the valve 1102. The balloon 304 can be, for example, a blocking balloon. In an implementation, the valve 1102 can be selectively removable from the graft device 302 (e.g., the valve 1102 can clip onto the end of the graft device 302, etc.). In another implementation, the valve 1102 can be affixed (e.g., permanently affixed, fastened to the end of graft device 302, etc.). The valve 1102 can be, for example, a heart valve (e.g., an artificial heart valve). In one example, the valve 1102 can be a bioprosthetic valve (e.g., the value 1102 can comprise tissue membrane, biological tissue, etc.). In another example, the valve 1102 can be a mechanical valve. The valve 1102 can comprise, for example, a wire mesh, metal alloy, pyrolytic carbon, chained polyester, another type of synthetic material, etc. In a non-limiting example, the valve 1102 can be a sutureless aortic valve and/or the graft device 302 can be an aortic graft. However, it is to be appreciated that the valve 1102 can be another type of valve and/or can comprise another type of material known in the art. It is also to be appreciated that the valve 1102 can be attached to the graft device 302 at a different location (e.g., at a different end of the graft device 302, at an inner portion of the graft device 302, etc.).

In certain implementations, the graft device 302 that comprises the valve 1102 can be employed with another graft device (e.g., the second graft device 1012 as shown in FIG. 10). In one example, the other graft device (e.g., the second graft device 1012) can be configured without a valve. In another example, the other graft device (e.g., the second graft device 1012) can also comprise a valve (e.g., a valve 1102). In a non-limiting example, a first graft device can be configured to be inserted into a vessel, where the first graft device comprises a valve attached to the first graft device. The valve can be attached to an end (e.g., a distal end) of the first graft device. A first blocking balloon can be configured to temporarily restrict passage of fluid or gas received via a first perfusion port when the first blocking balloon is temporary inserted into the first graft device. Furthermore, a second graft device can be configured to be inserted into the vessel or another vessel. A second blocking balloon can be configured to temporarily restrict the passage of the fluid or the gas when the second blocking balloon is temporary inserted into the second graft device. In one example, the second blocking balloon can be associated with a second perfusion port (e.g., a second perfusion port operatively connected to the first perfusion port and/or configured to allow passage of the fluid or the gas through the second blocking balloon, etc.). In another example, the second blocking balloon can be employed without a second perfusion port. In an implementation, a blocking balloon can be inserted into a graft device during a first procedure, and a valve can be attached to the graft device during a second procedure (e.g., after the blocking balloon is removed from the graft device).

In view of the example aspects shown and described herein, methods that may be implemented in accordance with the one or more of the disclosed aspects, will be better understood with reference to the following flow charts. While, for purposes of simplicity of explanation, the methods are shown and described as a series of blocks, it is to be understood that the disclosed aspects are not limited by the number or order of blocks, as some blocks may occur in different orders and/or at substantially the same time with other blocks from what is depicted and described herein. Moreover, not all illustrated blocks may be required to implement the methods described hereinafter.

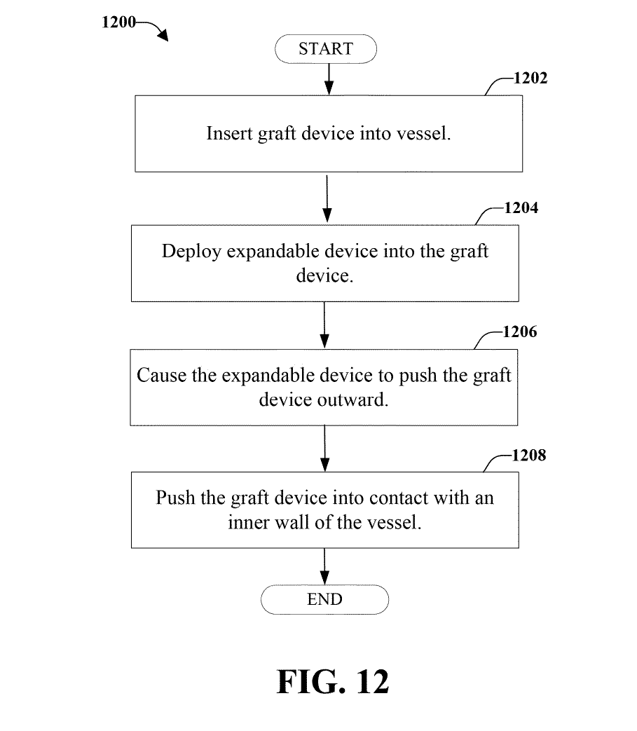

FIG. 12 illustrates a method 1200 for aortic dissection repair, according to an aspect. Method 1200 starts, at 1202, when a graft device is inserted into a vessel. The graft device can be a fixed length, however, according to some aspects, the graft device can include an expandable portion that can be rolled away from the graft device in order to lengthen the graft device.

At 1204, an expandable device is deployed into the graft device. The expandable device can be, for example, a balloon. At 1206, the expandable device pushes the graft device outward, toward the vessel. The graft device is pushed into contact with an inner wall of the vessel, at 1208.

In an implementation, the method 1200 can include unrolling a first end of the graft device and contacting the first end of the graft device with at least a portion of the vessel. For example, the graft device can include an expandable portion that can be unrolled to provide for a larger amount of coverage over the vessel.

According to another implementation, the graft device comprises at least one connector. Further to this implementation, pushing the graft device into contact with the inner wall of the vessel comprises attaching the at least one connector to the inner wall of the vessel. For example, the at least one connector can be a hook that grabs or impales the vessel when the graft device is pressed against the vessel. In another example, the at least one connector can be a sealant material strip or an adhesive strip that attaches to the vessel when the graft device is pressed against the vessel. Thus, the graft device is secured to the vessel, mitigating the need to hand sew the graft device in place.

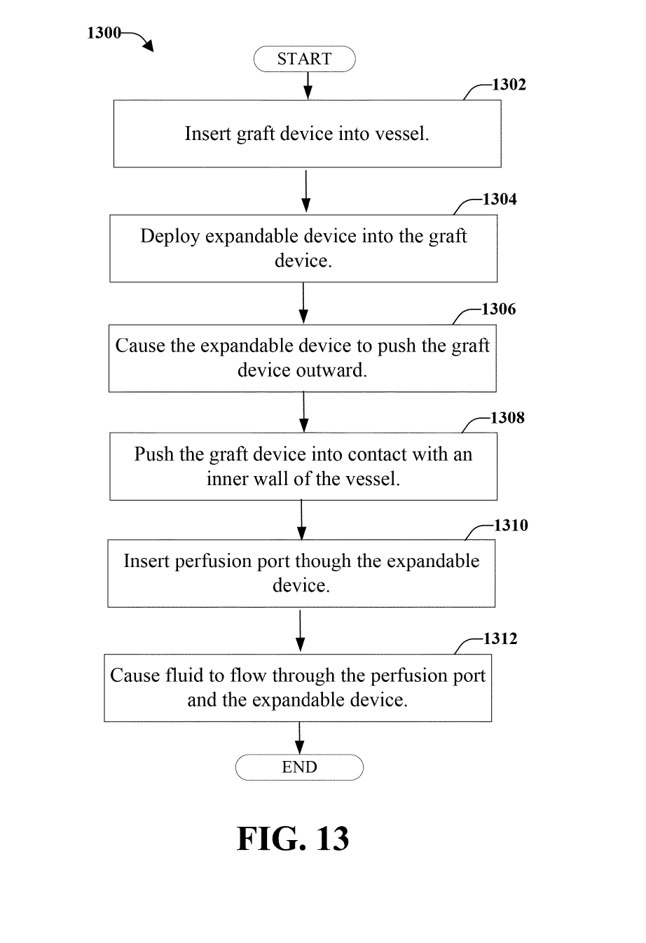

FIG. 13 illustrates another method for aortic dissection repair, according to an aspect. Method 1300 starts, at 1302, when a graft device of a fixed or variable length is inserted into a vessel. At 1304, an expandable device (e.g., a balloon) is deployed into the graft device. At 1306, the expandable device pushes the graft device outward, toward the vessel. The graft device is pushed into contact with an inner wall of the vessel, at 1308.

At 1310, a perfusion port is inserted through the expandable device. The perfusion port can be connected to a heart lung machine, for example. At 1312, fluid is caused to flow through the perfusion port and the expandable device. For example, before the perfusion port is connected to the heart lung machine, a valve is closed and blood cannot flow from the heart lung machine. When the valve is opened, the blood flows from the heart lung machine, through the perfusion port, and into the patient.

According to an implementation, the perfusion port comprises a first end located on a first side of the expandable device and a second end located on a second side of the expandable device. Further to this implementation, the method 1300 can include attaching another vessel (the vessel from which blood is being drawn from the patient's body) to a fluid collection device and attaching the fluid collection device to the first end of the perfusion port. Further, the method 1300 can include allowing the fluid to flow from the fluid collection device to the first end, past the expandable device, and out the second end to the another vessel.

In accordance with another implementation, the method 1300 can include inserting another graft device into another vessel and deploying another expandable device into the another graft device. Further to this implementation, the method 1300 includes causing the another expandable device to push the another graft device outward and pushing the another graft device into contact with the inner wall of the vessel.

According to another implementation, the method 1300 can include inserting a perfusion port through the expandable device and causing fluid to flow through the perfusion port and through the expandable device. Method 1300 can also include inserting another perfusion port through another expandable device and attaching a first end of the perfusion port to another first end of another perfusion port. Further, the method 1300 can include causing the fluid to flow from the first end of the perfusion port to the first end of the another perfusion port.

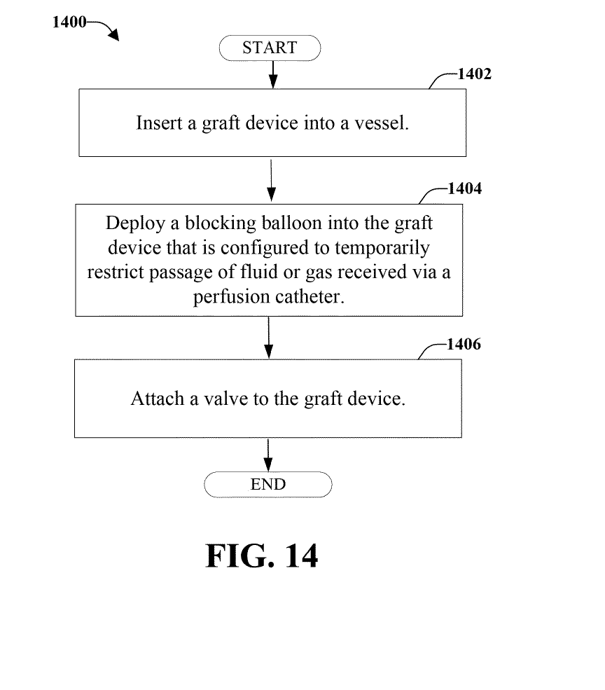

FIG. 14 illustrates yet another method for aortic dissection repair, according to an aspect. Method 1400 starts, at 1402, when a graft device of a fixed or variable length is inserted into a vessel. At 1404, a blocking balloon is deployed into the graft device. The blocking balloon is configured to temporarily restrict passage of fluid or gas received via a perfusion catheter. At 1406, a valve is attached to the graft device. The valve can be attached, for example, at a distal end of the graft device. In one example, the valve can be a wire mesh valve comprising tissue membrane. In another example, the valve can be a mechanical valve. In yet another example, the valve can be a tissue valve. However, it is to be appreciated that the valve can be a different type of valve. In an aspect, the valve can be attached to the graft device after the block balloon is deployed into the graft device and then removed from the graft device. For example, according to an implementation, the method 1400 can include removing the blocking balloon from the graft device.

According to an implementation, the method 1400 can include removing the valve from the graft device. The method 1400 can also include inserting another graft device into the vessel and/or another vessel. Additionally or alternatively, the method 1400 can include deploying another blocking balloon into the graft device and/or another graft device.

What has been described above includes examples that provide advantages of the one or more aspects. It is, of course, not possible to describe every conceivable combination of components or methods for purposes of describing the aspects, but one of ordinary skill in the art may recognize that many further combinations and permutations are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form.

What is claimed is:

1. A system, comprising:
a graft device configured to be inserted into a vessel;
a balloon configured to be inserted into the graft device and to temporarily restrict passage of fluid or gas received via a perfusion port when the balloon is inflated via a bulb that provides air pressure to the balloon, wherein the perfusion port is operatively connected to another perfusion port configured to allow the fluid or the gas to pass through another balloon; and
a valve configured to be attached to an end of the graft device.

2. The system of claim 1, wherein the valve is configured to be attached at a distal end of the graft device.

3. The system of claim 1, wherein the valve is configured to open and close to provide unidirectional flow of the fluid or the gas.

4. The system of claim 1, wherein the valve is selectively removable from the graft device.

5. The system of claim 1, wherein the valve is a wire mesh valve attached to the end of the graft device.

6. The system of claim 1, wherein the valve is a mechanical valve attached to the end of the graft device.

7. The system of claim 1, wherein the valve is a bioprosthetic valve attached to the end of the graft device.

8. The system of claim 1, wherein the bulb is configured to provide the air pressure to the balloon through a balloon infusion port.

9. The system of claim 1, further comprising:
another graft device configured to be inserted into another vessel, wherein the other balloon is configured to temporarily restrict passage of the fluid or the gas through the other vessel when the other balloon is temporary inserted into the other graft device.

10. The system of claim 9, wherein the other vessel is at least a portion of the vessel.

11. The system of claim 1, wherein the vessel is one of an ascending aorta, a descending aorta, or an arch of an aorta.

12. A system, comprising:
a graft device configured to be inserted into a vessel;
a valve configured to be attached to an end of the graft device;
a blocking balloon configured to be inserted into the graft device and to temporarily restrict passage of fluid or gas received via a perfusion catheter when the blocking balloon is inflated via a bulb that provides air pressure to the blocking balloon; and
another blocking balloon configured to temporarily restrict passage of the fluid or the gas received via another perfusion catheter when the other blocking balloon is inflated via another bulb that provides air pressure to the other blocking balloon, wherein the perfusion catheter is operatively connected to the other perfusion catheter.

13. The system of claim 12, further comprising another graft device configured to be inserted into another vessel.

14. The system of claim 12, wherein the valve comprises tissue membrane.

15. The system of claim 12, wherein the valve is a mechanical valve attached to the end of the graft device.

16. The system of claim 12, wherein the valve is wire mesh valve attached to the end of the graft device.

17. The system of claim 12, wherein the bulb is configured to provide the air pressure to the blocking balloon through a balloon infusion port.

18. A system comprising:
a first graft device configured to be inserted into a vessel, wherein the first graft device comprises a valve attached to an end of the first graft device;
a first blocking balloon configured to be inserted into the first graft device and to temporarily restrict passage of fluid or gas received via a first perfusion port when the first blocking balloon is inflated via a first bulb that provides air to the first blocking balloon;
a second graft device configured to be inserted into the vessel or another vessel; and
a second blocking balloon configured to be inserted into the second graft device and to temporarily restrict the passage of the fluid or the gas when the second blocking balloon is inflated via a second bulb that provides air to the second blocking balloon, wherein the first perfusion port is operatively connected to a second perfusion port associated with the second blocking balloon.

19. The system of claim 18, wherein the valve is attached to an end of the first graft device.

20. The system of claim 18, wherein the first bulb is configured to provide the air to the first blocking balloon through a first balloon infusion port and the second bulb is configured to provide the air to the second blocking balloon through a second balloon infusion port.
